(19) United States
(12) Patent Application Publication
(10) Pub. No.: US 2002/0005202 A1
(43) Pub. Date: Jan. 17, 2002

(54) Inflation indicators

(75) Inventor: Andrew Martin Parry, Dymchurch (GB)

Correspondence Address: Connolly Bove Lodge & Hutz LLP, Suite 800, 1990 M Street, N.W., Washington, DC, 20036-3425, US

(73) Assignee: Smiths Group plc, London (GB)

(21) Appl. No.: 09/949,626

(22) Filed: Sep. 12, 2001

Related U.S. Application Data
(63) Continuation of application No. 09/435,720, filed on Nov. 8, 1999.

(30) Foreign Application Priority Data
Nov. 27, 1998 (GB) 9825881.7

(51) Int. Cl. A61M 16/00

(52) U.S. Cl. 128/207.15

(57) ABSTRACT

An inflation indicator balloon for a cuffed tracheal tube is moulded directly onto an and of an inflation line. The inflation line is slipped on one end of a core pin and a parison is injection moulded in a first cavity with one end of the parison on top of the inflation line. The parison is then blow moulded to the desired shape of the inflation balloon in a second cavity. The balloon and line are removed from the core pin and a valve is inserted in the end oposite the inflation line.

### BACKGROUND OF THE INVENTION

This invention relates to inflation indicators and their manufacture.

Cuffed medical tubes, such as tracheal tubes, are provided with some form of inflation indicator so that a clinician can determine whether the cuff is inflated or deflated when the tube is located in the body. The inflation indicator may be a flat balloon joined at one end with a small bore inflation line and at the other end with a connector and valve by which a syringe or similar device is connected to the inflation line. When the cuff of the tube is inflated, pressure is communicated to the inflation indicator, which is inflated to a more circular section. Conventionally, the indicator balloon is blow moulded and subsequently assembled onto and attached to the inflation line, the valve/connector and to its dust cap. Blow moulding a shape of this kind tends to lead to a relatively large amount of waste material and the assembly operations also add to the cost of the final product. It can also be a problem joining the balloon to the inflation line without leaking because the flat shape of the balloon does not lend itself to forming an effective seal around the cylindrical surface of the inflation line. Examples of inflation indicators are described in, for example, U.S. Pat. Nos. 3,810,474, 4,018,231, 4,130,617, 4,178,939, GB 2164565, GB 2174303 and GB 2213592.

### BRIEF SUMMARY OF THE INVENTION

It is an object of the present invention to provide an alternative inflation indicator and method of manufacture.

According to one aspect of the present invention there is provided an inflation indicator for a cuffed medico-surgical tube, the indicator including an inflatable balloon attached at one end to an inflation line, the balloon being moulded to its final shape with the inflation line extending in the one end of the balloon so that the balloon is moulded onto the inflation line.

The balloon may have an inflatable region that is generally flat and may include a valve assembly disposed in an opposite end of the inflatable balloon. The balloon preferably includes a dust cap moulded with it.

According to another aspect of the present invention there is provided a method of manufacture of an inflation indicator comprising the steps of forming a parison of generally tubular shape about an end of an inflation line, then moulding the parison to the shape of an inflation balloon with the inflation line in situ such that the end of the inflation line is attached sealingly with one end of the balloon.

The inflation line is preferably fitted on one end of a core pin and the parison is preferably moulded in a first cavity on the core pin about an end of the inflation line. The parison is preferably moulded on the core pin to the shape of the inflatable balloon in a second cavity. The parison may be formed by injection moulding and be moulded to the shape of the inflation balloon by blow moulding. The method may include the subsequent step of inserting a valve in the opposite end of the balloon. The parison may be moulded with an integral dust cap.

According to a further aspect of the present invention there is provided an inflation indicator made by a method according to the above other aspect of the present invention.

According to a fourth aspect of the invention, there is provided a cuffed medico-surgical tube including an inflation indicator according to the above one or further aspect of the present invention.

A cuffed tracheostomy tube with an inflation indicator, and its method of manufacture, will now be described, by way of example, with reference to the accompanying drawings.

### BRIEF DESCRIPTION OF THE DRAWINGS

FIG. 1 is a side elevation view of the tube;

FIG. 2 is an enlarged side elevation view of the inflation indicator;

FIG. 3 is an enlarged sectional view along the line III-III of FIG. 2;

FIG. 4 is an enlarged transverse sectional view along the line IV-IV of FIG. 2;

FIG. 5 is a sectional side elevation view of a first station of the machine used to preform the inflation indicator;

FIG. 6 is an enlarged sectional side elevation view of the pre-form produced by the machine shown in FIG. 5; and

FIG. 7 is a sectional side elevation view of a second station of the machine used to form the inflation indicator.

### DETAILED DESCRIPTION OF THE PREFERRED EMBODIMENT

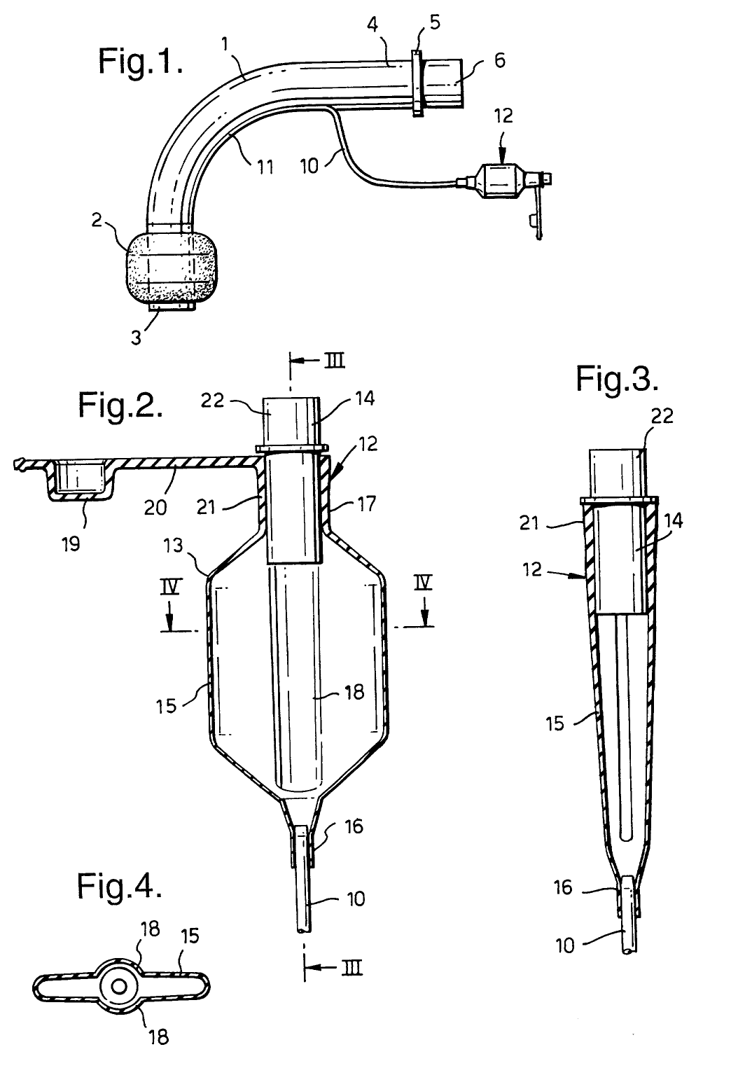

With reference first to FIGS. 1 to 4, the tracheostomy tube includes a curved, moulded shaft 1 of circular section with an inflatable cuff 2 surrounding the shaft close to the patient end 3. The machine end 4 of the tube has an integrally moulded flange 5 and connector 6. The cuff2 is inflated and deflated by means of a small bore inflation line 10 secured in a channel 11 extending along the outside of the shaft 1 or via an extruded lumen within the wall of the tube. The patient end of the inflation line 10 extends within and opens into the interior of the cuff 2. The machine end of the inflation line 10 is provided with an inflation indicator 12.

The inflation indicator 12 includes a moulded balloon 13 and a separate valve/connector assembly 14. The balloon 13 includes an inflatable region 15, a patient end portion 16 joined with the inflation line 10, and a machine end portion 17 joined with the valve assembly 14. The inflation balloon 13 is of PVC and is moulded in a two-stage process of injection and blow moulding, which will be described in greater detail later. The inflatable region 15 is of generally rectangular shape with tapered ends and is substantially flat (in its uninflated state), with a raised rib 18 extending longitudinally in the centre of each side to ensure a continuous gas path through the indicator, even when a negative pressure is applied (as described in greater detail in GB 2213592). The balloon 13 is about 50 mm long, with the inflatable region 15 being about 35 mm long and 20 mm wide. The wall thickness of the balloon 13 along the inflatable region 15 and the patient end 16 is about 0.5 mm, whereas at the machine end, the wall is about 2 mm thick. The balloon 13 is moulded with an integral dust cap 19 attached with the machine end of the indicator by a flexible web 20 about 25 mm long.

The patient end portion 16 is in the form of a short collar of cylindrical shape, with an internal diameter of about 1.4 mm and an external diameter of 2.4 mm. The collar 16 is moulded about and bonded to the machine end of the inflation line 10, as will be described in greater detail later. At its machine end 17, the balloon 13 has a larger diameter sleeve 21, with an internal diameter of about 7 mm. The valve-assembly 14 is conventional and is bonded into the sleeve 21, the external end of the valve assembly having a female connector 22 shaped to allow the nose of a syringe to be inserted. The connector 22 is shaped to allow the nose of the syringe to contact and displace a valve element (not shown) from a valve seat within the assembly 14 so as to open a gas flow passage through the valve. At the same time, the nose of the syringe forms a seal with the connector 22.

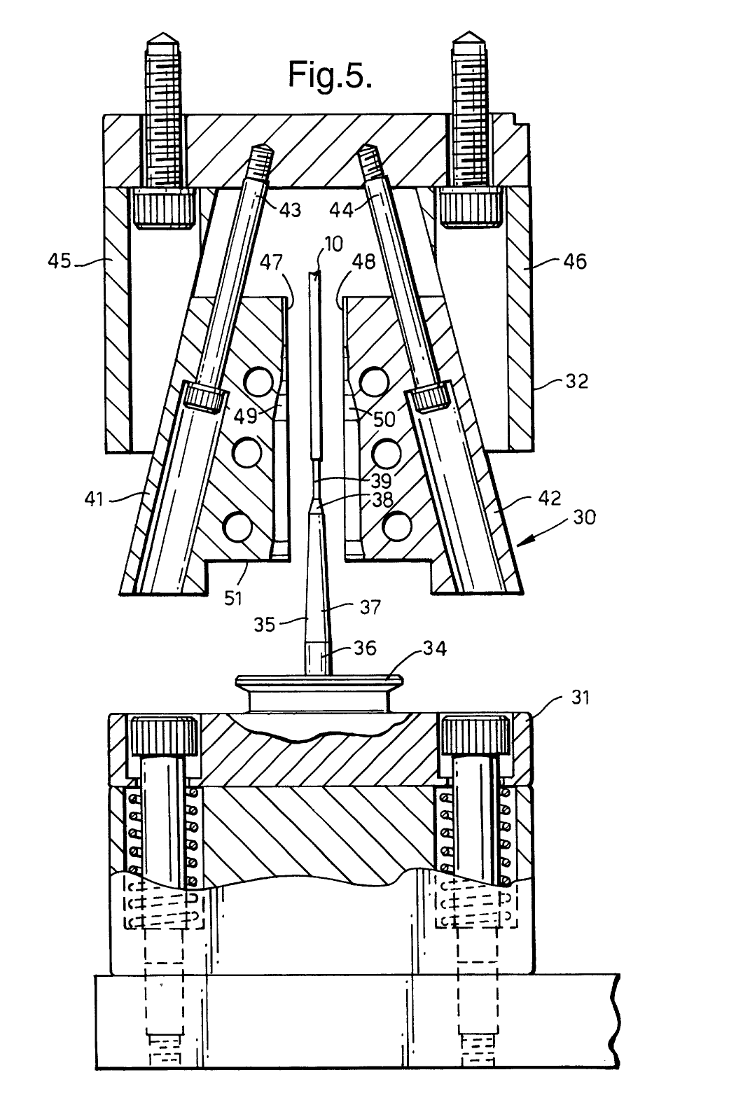

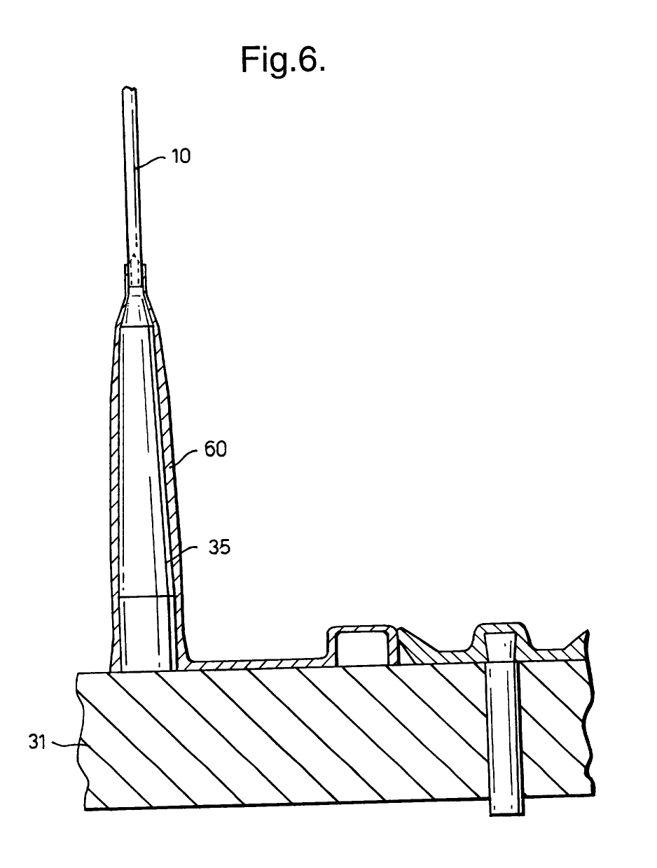

With reference now to FIGS. 5 to 7, the inflation indicator balloon 13 is made in two stages on a moulding machine 30 having a base assembly 31 and two different upper stations 32 and 33. As shown in FIG. 5, the base assembly 31 comprises a base 34 and a vertically-extending, hollow and porous core pin 35, the external shape of which defines the internal shape of a preform of the indicator. More particularly, the core pin 35 has a lower section 36 of cylindrical shape and diameter of 7 mm, an intermediate section 37, which tapers at a shallow angle to a reduced diameter to a more steeply tapered shoulder 38, which joins with a small diameter upper section 39. The upper section 39 receives the machine end of the inflation line 10 pushed onto the core pin 35 by a distance of about 4 mm. The first upper station 32 is an injection moulding station and has two mould parts 41 and 42 that are slidable down and outwardly along angled pins 43 and 44 extending parallel to respective heel blocks 45 and 46. The mould parts 41 and 42 have respective opposed, vertical faces 47 and 48 shaped with recesses or cavities 49 and 50 to define a cavity for the external shape of the balloon preform 60 (FIG. 6). More particularly, the cavities 49 and 50 conform substantially to the profile of the core pin 35 except that the spacing between the recesses and the core pin allows for a thicker layer of material along the intermediate section 37 than along the shoulder 38 and upper section 39. The lower surface 51 of one of the mould parts 41 is formed with a recess and the base 34 is formed with a projection (neither shown) which together define the dust cap 19 and web 20 of the balloon 13.

In operation, the upper station 32 is lowered about the base assembly 31 and the two mould parts 41 and 42 are closed around the core pin 35 with the inflation line 10 in place. Molten PVC is then injected into the cavity between the mould parts 41 and 42 and the core pin 35. The upper station 32 is then lifted and the mould parts 41 and 42 are separated, leaving a preform or parison 60 of the kind shown in FIG. 6 on the base assembly 31, with an integral, moulded dust cap.

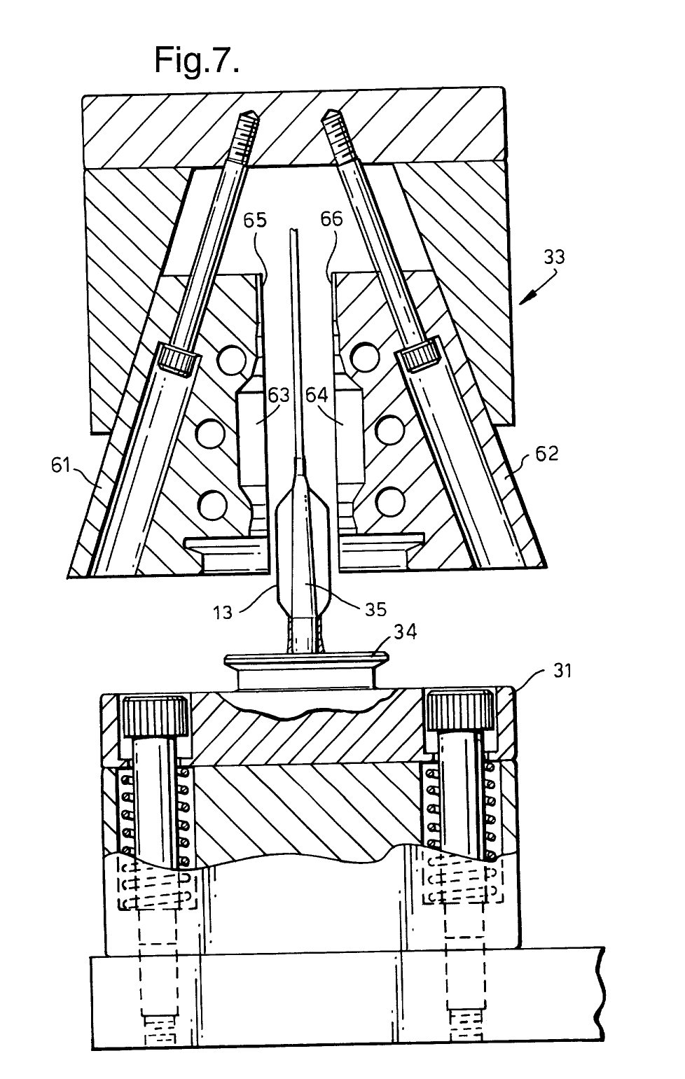

The base assembly 31 is then moved on to the second, blow station 33 (FIG. 7), which also has two mould parts 61 and 62 with recesses or cavities 63 and 64 in their respective opposed vertical faces 65 and 66. These cavities 63 and 64 are shaped with the external profile of the final shape of the balloon 13, that is, they define the generally flat shape of the indicator balloon.

The blow station 33 is lowered and the mould parts 61 and 62 are closed about the core pin 35 and preform 60. The mould parts 61 and 62 are heated and pressure is applied via the core pin 35 to the inside of the preform 60 so that this is blown outwardly to fill the shape of the cavities 63 and 64. The upper station 33 is then raised and the mould parts 61 and 62 are separated allowing the balloon 13 in its final form to be removed from the core pin 35. The indicator 12 is completed by inserting the valve assembly 14 into the sleeve 21 and bonding this in position such as by welding or adhesive. It may be necessary in some cases for the blow moulding stage to be carried out in two parts. The parison is first blow moulded to an intermediate shape at a first blow station and is then moved to a second blow station where it is blow moulded to its final shape. This helps ensure a more constant wall thickness over the indicator balloon.

It can be seen that, because the patient end of the balloon is moulded about the inflation line, there can be a perfect seal with the inflation line, which cannot always be guaranteed when previous assembly techniques are used. The manufacturing method described enables the number of manual assembly operations required to be greatly reduced. Assembly is also reduced because the dust cap can be moulded integrally with the balloon, rather than being made separately and subsequently assembled. The amount of waste material produced by the injection blow moulding technique described can be less than previous blow moulding techniques. The two stage process of injection moulding and blow moulding also enables more accurate control of wall thickness of the final product.

What I claim is:

1. An inflation indicator for a cuffed medico-surgical tube, the indicator comprising an inflatable balloon and an inflation line, wherein said balloon is attached to one end of said inflation line, and wherein said balloon is moulded to its final shape with said inflation line extending in the said one end of said balloon so that said balloon is moulded onto said inflation line.

2. An inflation indicator according to claim 1, wherein said balloon has an inflatable region that is generally flat.

3. An inflation indicator according to claim 1, including a valve assembly disposed in one end of said inflatable balloon opposite said inflation line.

4. An inflation indicator according to claim 1, wherein said balloon includes a dust cap moulded with said balloon.

5. A cuffed medico-surgical tube comprising a tubular shaft, and inflatable cuff encircling said shaft towards one end and an inflation indicator for indicating inflation of said cuff, wherein said indicator comprises an inflatable balloon and an inflation line, said balloon being attached to one end of said inflation line, and wherein said balloon is moulded to its final shape with said inflation line extending in the said one end of said balloon so that said balloon is moulded onto said inflation line.

6. A method of manufacture of an inflation indicator comprising the steps of forming a parison of generally tubular shape about an end of an inflation line, then moulding said parison to a shape of an inflation balloon with said inflation line in situ such that said end of said inflation line is attached sealingly with one end of said balloon.

7. A method according to claim 6, wherein said inflation line is fitted on one end of a core pin and said parison is moulded in a first cavity on said core pin about an end of said inflation line.

8. A method according to claim 7, wherein said parison is moulded on said core pin to the shape of said inflation balloon in a second cavity.

9. A method according to claim 6, wherein said parison is formed by injection moulding, and wherein said parison is moulded to the shape of said inflation balloon by blow moulding.

10. A method according to claim 6, wherein said parison is moulded with an integral dust cap.

11. A method according to claim 7, including the subsequent step of removing said balloon from the core pin and inserting a valve in an end of said balloon opposite said inflation line.

12. A method of manufacture of an inflation indicator comprising the steps of: fitting one end of an inflation line on an end of a core pin; moulding a parison of generally tubular shape on said core pin in a first cavity about the end of said inflation line; subsequently blow moulding said parison in a second cavity to a shape of an inflation balloon; and then removing the inflation balloon with said inflation line attached from said second cavity and from said core pin.
